(19) United States
(12) Patent Application Publication
(10) Pub. No.: US 2022/0370001 A1
(43) Pub. Date: Nov. 24, 2022

(54) INTESTINAL DIAGNOSTIC SCREENING DEVICE AND METHOD FOR TARGETED GASTROINTESTINAL THERAPY

(71) Applicant: AJM MED-I-CAPS LTD, Strovolos, Nicosia (CY)

(72) Inventors: Anastasios Koulaouzidis, Strovolos, Nicosia (CY); Julius Georgiou, Strovolos, Nicosia (CY); Maya Thanou, Strovolos, Nicosia (CY)

(21) Appl. No.: 17/638,295

(22) PCT Filed: Aug. 26, 2020
(86) PCT No.: PCT/IB2020/057979

§ 371 Date: Feb. 25, 2022

Related U.S. Application Data
(60) Provisional application No. 62/891,769, filed on Aug. 26, 2019.

(51) Int. Cl. A61B 5/00; A61B 1/04; A61B 1/00; A61K 49/00; A61K 31/704

(52) U.S. Cl. CPC A61B 5/4216; A61B 5/4839; A61B 1/041; A61B 5/6861; A61B 5/6886; A61B 5/0017; A61B 5/0071; A61B 1/00156; A61B 1/043; A61K 49/0084; A61K 31/704; A61K 49/0093; A61B 2560/0214

(57) ABSTRACT

A tracer detection device includes an enclosed body, and a plurality of tracer sensors, a battery, a memory, and a transmitter, each disposed within the enclosed body. The plurality of tracer sensors is configured to detect measurement values at a surface and underneath the surface of a gastrointestinal tract. The battery is configured to power the plurality of tracer sensors. The memory is configured to receive measurement values detected by the plurality of tracer sensors. The transmitter is configured to transmit measurement values detected by the plurality of tracer sensors to an external device after the enclosed body has passed through the gastrointestinal tract. The enclosed body includes a steering feature that ensures the enclosed body is oriented in an intended direction. The plurality of tracer sensors triggers release of a drug. The plurality of tracer sensors estimate distances to gastrointestinal walls for normalizing signals.

### PRIORITY CLAIM

This application claims priority to and the benefit of U.S. Provisional Patent Application No. 62/891,769 entitled INTESTINAL DIAGNOSTIC SCREENING DEVICE AND METHOD FOR TARGETED GASTROINTESTINAL THERAPY, filed on Aug. 26, 2019, the entire contents of which are incorporated by reference and relied upon.

### BACKGROUND

With gastrointestinal (GI) diseases such as neoplasia, specifically cancer in the stomach, in the small intestine, and/or the colon, early detection is critical, as it drastically improves the survival rate for patients. Unfortunately, many patients still consider current detection methods and procedures invasive, intimidating, often undesirable and/or uncomfortable. Eventually, patients are discouraged from examinations with great potential for early lesion detection and curative treatment.

One typical lesion detection procedure is the colonoscopy. However, patients may have a strong aversion to laxative purge and/or endoscope insertion. Furthermore, colonoscopies are typically limited to large intestine analysis only; the majority of the small-intestine, but for a few inches of the terminal ileum, is not normally accessible with this procedure. Another detection procedure is fecal sample testing. However, patients usually have a strong aversion to self-collection of fecal matter. An alternate to these detection procedures, which avoids many of the negative characteristics, is the swallowable capsule.

With swallowable imaging capsules, the typical procedure includes the patient swallowing a capsule that records images of the GI tract, such as the stomach, the small intestine, and the colon. The visual footage is dynamically transmitted to a patient receiver, housed in a wearable belt, as the capsule is travels through the GI tract. An expert gastroenterologist or other experienced reader then watches the entire recorded footage, in order to affirmatively identify any areas of concern. The data must be dynamically transmitted to an external device, because storing high-resolution video in the capsule for the entire time to transverse the entire GI tract is not feasible, both in terms of data storage capacity and in terms of battery energy requirements.

The above diagnostic procedures and methods are either undesirable/unpleasant to patients and intimidating (if not physically uncomfortable in the case of colonoscopy), and/or very time consuming for a physician to examine in the case of video capsules, thus making them too costly for a screening test. Improved devices, systems, and methods for GI disease screening, diagnosis, and non-invasive therapy are therefore needed.

### SUMMARY

The devices, systems, and methods disclosed herein improve the detection paradigm associated with screening and diagnosis of GI tract pathologies, including GI tract neoplasia (benign or malignant) including cancer precursors, GI cancers, and other inflammatory conditions such as inflammatory bowel disease (IBD). This platform implements both a chemical entity with affinity to precancerous and cancerous cells called a tracer and an ingestible tracer detection system, to allow for early pathology detection and subsequent treatment.

In light of the disclosure herein, and without limiting the scope of the invention in any way, in a first aspect of the present disclosure, which may be combined with any other aspect listed herein unless specified otherwise, a tracer detection device includes an enclosed body, a plurality of tracer sensors, a battery, a memory, and a transmitter. The plurality of tracer sensors is disposed within the enclosed body and configured to detect measurement values both at a surface and underneath the surface of a gastrointestinal tract. The battery is disposed within the enclosed body and configured to power the plurality of tracer sensors. The memory is disposed within the enclosed body and configured to receive measurement values detected by the plurality of tracer sensors. The transmitter is disposed within the enclosed body and configured to transmit measurement values detected by the plurality of tracer sensors to an external device after the enclosed body has passed entirely through the gastrointestinal tract. The enclosed body further includes a steering feature that ensures the enclosed body is oriented in an intended direction while passing through the gastrointestinal tract. The plurality of tracer sensors is further configured to trigger release of a drug within the gastrointestinal tract. The plurality of tracer sensors further estimate distances between the enclosed body and walls of the gastrointestinal tract for normalizing signals.

In a second aspect of the present disclosure, which may be combined with any other aspect listed herein unless specified otherwise, the plurality of tracer sensors is configured to detect a plurality of tracer spikes of a tracer.

In a third aspect of the present disclosure, which may be combined with any other aspect listed herein unless specified otherwise, the plurality of tracer spikes is associated with a plurality of discrete locations in the gastrointestinal tract.

In a fourth aspect of the present disclosure, which may be combined with any other aspect listed herein unless specified otherwise, the measurement values detected by the plurality of tracer sensors comprise a series of data points including the plurality of spikes.

In a fifth aspect of the present disclosure, which may be combined with any other aspect listed herein unless specified otherwise, the steering feature is at least one of a long flexible tail and an expandable polymer.

In a sixth aspect of the present disclosure, which may be combined with any other aspect listed herein unless specified otherwise, the battery is a silver oxide battery.

In a seventh aspect of the present disclosure, which may be combined with any other aspect listed herein unless specified otherwise, the tracer detection device further includes a hyperspectral camera, configured to selectively capture pictures or video of the gastrointestinal tract when triggered by a tracer signal.

In an eighth aspect of the present disclosure, which may be combined with any other aspect listed herein unless specified otherwise, the plurality of tracer sensors trigger release of the drug within the gastrointestinal tract via at least one of light, hyperthermia, ultrasound, or pH change.

In a ninth aspect of the present disclosure, which may be combined with any other aspect listed herein unless specified otherwise, the external device is one of a smart-watch, wristband, cellphone, tablet, or laptop.

In a tenth aspect of the present disclosure, which may be combined with any other aspect listed herein unless specified otherwise, a tracer lesion detection system includes a tracer and a detection device. The detection device includes a plurality of tracer sensors, a battery, a memory, and a transmitter. The plurality of tracer sensors is configured to detect measurement values both at a surface and underneath the surface of a gastrointestinal tract. The battery is configured to power the plurality of tracer sensors. The memory is configured to receive measurement values detected by the plurality of tracer sensors. The transmitter is configured to transmit measurement values detected by the plurality of tracer sensors to an external device after the enclosed body has passed entirely through the gastrointestinal tract. The detection device further includes a steering feature that ensures the detection is oriented in an intended direction while passing through the gastrointestinal tract. The plurality of tracer sensors is further configured to trigger release of a drug within the gastrointestinal tract. The plurality of tracer sensors further estimate distances between the enclosed body and walls of the gastrointestinal tract for normalizing signals.

In an eleventh aspect of the present disclosure, which may be combined with any other aspect listed herein unless specified otherwise, the plurality of tracer sensors is configured to detect a plurality of tracer spikes of the tracer.

In a twelfth aspect of the present disclosure, which may be combined with any other aspect listed herein unless specified otherwise, the plurality of tracer spikes is associated with a plurality of discrete locations in the gastrointestinal tract.

In a thirteenth aspect of the present disclosure, which may be combined with any other aspect listed herein unless specified otherwise, the measurement values detected by the plurality of tracer sensors comprise a series of data points including the plurality of spikes.

In a fourteenth aspect of the present disclosure, which may be combined with any other aspect listed herein unless specified otherwise, the steering feature is at least one of a long flexible tail and an expandable polymer.

In a fifteenth aspect of the present disclosure, which may be combined with any other aspect listed herein unless specified otherwise, the battery is a silver oxide battery.

In a sixteenth aspect of the present disclosure, which may be combined with any other aspect listed herein unless specified otherwise, the detection device further includes a hyperspectral camera, configured to selectively capture images or video of the gastrointestinal tract when triggered by a tracer signal.

In a seventeenth aspect of the present disclosure, which may be combined with any other aspect listed herein unless specified otherwise, the plurality of tracer sensors trigger release of the drug within the gastrointestinal tract via at least one of light, hyperthermia, ultrasound, or pH change.

In an eighteenth aspect of the present disclosure, which may be combined with any other aspect listed herein unless specified otherwise, the external device is one of a smartwatch, wristband, cellphone, tablet, or laptop.

In a nineteenth aspect of the present disclosure, which may be combined with any other aspect listed herein unless specified otherwise, the tracer includes at least one of near-infrared fluorescence (NIRF) labeled liposomal nanoparticles and NIRF nanovesicles.

In a twentieth aspect of the present disclosure, which may be combined with any other aspect listed herein unless specified otherwise, the NIRF-labeled liposomal nanoparticles comprises a mixture of lipids selected from the group consisting of 1,2-Dipalmitoyl-sn-glycero-3-phosphocholine (DPPC; 16:0 PC), 1,2-Distearoyl-sn-glycero-3-phosphocholine (DSPC 18:0), (ω-methoxy-polyethylene glycol 2000)-N-carboxy-1,2-distearoyl-sn-glycero-3-phosphoethanolamine (PEG²⁰⁰⁰-DSPE) and combinations thereof, and a NIRF labelling agent selected from the group consisting of IRDye® 800CW-DSA, near-infrared (NIR) nanoparticles and combinations thereof, where the IRDye® 800CW-DSA is a conjugate of IRDye® 800CW with a lipid of N,N-Distearylamidomethylamine (DSA).

In a twenty-first aspect of the present disclosure, which may be combined with any other aspect listed herein unless specified otherwise, the NIRF-labeled liposomal nanoparticles further comprises a drug and/or an active pharmaceutical ingredient (API).

In a twenty-second aspect of the present disclosure, which may be combined with any other aspect listed herein unless specified otherwise, the drug or active pharmaceutical ingredient (API) is chemotherapeutic such as doxorubicin.

In a twenty-third aspect of the present disclosure, which may be combined with any other aspect listed herein unless specified otherwise, the tracer is NIRF labeled polymeric nanoparticles.

In a twenty-fourth aspect of the present disclosure, which may be combined with any other aspect listed herein unless specified otherwise, the MRF labeled polymeric nanoparticles are IRDye® CW800 labeled hyaluronic acid nanoparticles.

In a twenty-fifth aspect of the present disclosure, which may be combined with any other aspect listed herein unless specified otherwise, the MRF labeled polymeric nanoparticles are IRDye® CW800 labeled Albumin nanoparticles.

In a twenty-sixth aspect of the present disclosure, which may be combined with any other aspect listed herein unless specified otherwise, the tracer is near infrared fluorescent quantum dots (NIRF-QDs).

In a twenty-seventh aspect of the present disclosure, which may be combined with any other aspect listed herein unless specified otherwise, the tracer is iron oxide magnetic nanoparticles selected from the group consisting of Fe₃O₄-PMA nanoparticles, [(ZnₓFe_{y})Fe₂O₄]-Polymer nanoparticles, and combinations thereof.

In a twenty-eighth aspect of the present disclosure, which may be combined with any other aspect listed herein unless specified otherwise, the tracer is administered to a patient orally or by intravenous (IV) injection.

In a twenty-ninth aspect of the present disclosure, which may be combined with any other aspect listed herein unless specified otherwise, a tracer detection method includes injecting a tracer into a circulatory system of a patient and introducing a detection device into a gastrointestinal tract of the patient. Introduction is performed by the patient swallowing the detection device. The method includes detecting, via the detection device, measurement values including a plurality of tracer spikes of the tracer both at a surface and underneath the surface of the gastrointestinal tract. The method includes transmitting, via the detection device, the measurement values including the plurality of tracer spikes to an external device after the enclosed body has passed entirely through the gastrointestinal tract, such that the plurality of tracer spikes may be associated with a plurality of discrete locations in the gastrointestinal tract. The detection device further includes a steering feature that ensures the detection is oriented in an intended direction while passing through the gastrointestinal tract. The plurality of tracer sensors is further configured to trigger release of a drug within the gastrointestinal tract. The plurality of tracer sensors further estimate distances between the enclosed body and walls of the gastrointestinal tract for normalizing signals.

In a thirtieth aspect of the present disclosure, which may be combined with any other aspect listed herein unless specified otherwise, a method for preparing NIRF-labeled liposomal nanoparticles for lesion detection includes the following steps: 1) synthesizing DSA; 2) mixing DSA with a solution of 1:1 dichloromethane and methanol to form a lipid solution; 3) dissolving IRDye® 800CW NHS ester in methanol then immediately combine with the lipid solution to form a mixture; 4) covering the mixture to protect from light and mixing for 0.5-5.0 hours; 5) reducing the solvent of the mixture in vacuum to form a dried mixture; 6) dissolving the dried mixture in minimal amount of chloroform to form a reacted solution; 7) applying the reacted solution to a silica micro-column in chloroform; and 8) reapplying unbound fraction of the reacted solution several times before elution and washing with the same solvent chloroform. Excess DSA and unreacted IRDye® 800CW NHS ester is retained on the column and the latter is estimated at <10% of the colored fraction. The unbound product is dried to a film and stored as a solution in 1 mL chloroform. This blue/green solution shows a strong fluorescence emission with Exmax 785 nm and Emmax 808 nm (reported values of 774 nm and 789 nm for the free dye).

In a thirty-first aspect of the present disclosure, which may be combined with any other aspect listed herein unless specified otherwise, a method for preparing iron oxide magnetic nanoparticles for cancer detection includes the following steps: 1) synthesizing Fe₃O₄ by thermal decomposition; 2) synthesizing [(ZnₓFe_{y})Fe₂O₄] by thermal degradation, where the ratio of x to y is from 1:1 to 1:10; 3) mixing Fe₃O₄ and [(ZnₓFe_{y}) Fe₂O₄] in the ratio between 1:1 and 1:10 to form a superparamagnetic iron nanoparticles (SPIONs); 4) dissolving PMAO (poly (maleic anhydride-alt-1-octadecene) in chloroform in a flask and stirring vigorously to obtain a clear solution; and 5) adding the SPIONs to the flask; stirring with a magnetic stirrer for 0.5-5 hours at room temperature in the sealed flask to obtain a translucent solution with a red-brown tint; placing the flask in a rotary evaporator for 5-60 minutes at room temperature to remove the chloroform; adding 0.5-5 mL chloroform to the flask to re-dissolve the SPIONs followed by adding 0.01-1.0M NaOH aqueous solution at 5-30 mL; agitating the flask whilst heating to 60° C. for 5-60 minutes; adding additional 5-20 mL of the NaOH solution whilst heating and agitating the flask to obtain a clear black solution showing no evidence of a biphasic system; centrifuging the clear black solution for 5-60 minutes to obtain the nanoparticles.

In a thirty-second aspect of the present disclosure, any of the structure, functionality, and alternatives discussed in connection with any of FIGS. 1 to 6 may be combined with any of the structure, functionality, and alternatives discussed in connection with any other one or more of FIGS. 1 to 6.

In light of the disclosure and aspects set forth herein, it is accordingly an advantage of the present disclosure to provide devices, systems, and methods of diagnostic screening that efficiently manage power consumption, by detecting the tracer concentration.

It is another advantage of the present disclosure to provide devices and systems that are omnidirectional and can thus tumble throughout the GI tract.

It is another advantage of the present disclosure to provide devices and systems that are semi-omnidirectional and include features to prevent tumbling in the large-intestine, thus saving power/space within the enclosure by halving the required number of tracer sensors.

It is yet another advantage of the present disclosure to provide customized integrated circuit devices and systems that efficiently manage memory storage and related transmission to external devices.

It is a further advantage of the present disclosure to provide devices and systems that are smaller in size, to ensure patient compliance with device administration.

It is yet a further advantage of the present disclosure to provide devices and systems that provide for follow up analysis, if required, such as via endoscopic assessment.

It is still a further advantage of the present disclosure to provide chemically stable diagnostic chemical entities ensuring appropriate time windows for fluorescent detection or through the use of electromagnetic waves.

Additional features and advantages of the disclosed devices, systems, and methods are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein. Moreover, it should be noted that the language used in the specification has been selected for readability and instructional purposes, and not to limit the scope of the inventive subject matter.

### BRIEF DESCRIPTION OF THE FIGURES

Understanding that figures depict only typical embodiments of the invention and are not to be considered to be limiting the scope of the present disclosure, the present disclosure is described and explained with additional specificity and detail through the use of the accompanying figures. The figures are listed below.

FIG. 1A is an elevation view of a tracer detection device, according to an example embodiment of the present disclosure.

FIG. 1B is a circuit block diagram of a detection device, according to an example embodiment of the present disclosure.

FIG. 1C is an elevation view of a tracer detection device with a tumble resisting tail and a semi-omnidirectional detection field.

FIG. 1D is an elevation view of a tracer detection device with a tumble resisting, expandable polymer feature and a semi-omnidirectional detection field.

FIG. 2A is a graph of NIRF absorbance and emission spectra of CF750 at varying wavelengths, according to an example embodiment of the present disclosure.

FIG. 2B is a graph of near-infra-red fluorescence absorbance and emission spectra of IRDye® CW800 at varying wavelengths, according to an example embodiment of the present disclosure.

FIG. 2C is a graph of absorbance and emission spectra of doxorubicin at varying wavelengths, according to an example embodiment of the present disclosure.

FIG. 3 is an illustration of exemplary chemical structures of a lipid N,N-Distearylamidomethylamine (DSA) and its conjugate with IRDye® CW800, according to an example embodiment of the present disclosure.

FIG. 4A illustrates a typical scheme of liposome structure with NIRF liposomal nanoparticles labeled with IRDye® CW800, according to an example embodiment of the present disclosure.

FIG. 4B illustrates a scheme of using NIRF polymeric nanoparticles (such as NIRF Hyaluronic acid and nanoparticles) as a tracer, according to an example embodiment of the present disclosure.

FIGS. 5A to 5C illustrate examples of in vivo particles in tumors, according to example embodiments of the present disclosure.

FIG. 6 illustrates an anatomical diagram of a detection device passing through a GI tract, according to an example embodiment of the present disclosure.

### DETAILED DESCRIPTION

Certain embodiments described herein relate generally to diagnostic screening of the GI tract. More particularly, some embodiments described herein relate to the detection of increased tracer concentrations (spikes) in abnormal GI tract tissue. These tracer spikes may identify GI abnormalities, including GI neoplasia such as polyps, cancer precursors, cancers, infectious GI conditions, or inflammatory GI conditions such as IBD. For example, the tracer may advantageously bond to particular abnormalities, such that the concentration of the tracer is higher at the abnormality. The tracer is chemically stable, to ensure usefulness for appropriate time windows such as typical digestion cycle time. By detecting a spike in tracer, related to an increase in tracer concentration, abnormalities are associated with discrete physical locations along the GI tract. By identifying the presence at a discrete physical location, the physician may perform additional follow up analysis, such as endoscopic assessment. For example, endoscopic assessment may be performed in a targeted manner, with respect to the discrete physical locations identified.

This detection paradigm improves power management and consumption, ultimately allowing the screening of the entire intestine with one detection device without running out of battery-life, as detecting tracer spikes requires far less power than other means of diagnosis, such as continuous streaming of photography or video. By associating tracer spikes with discrete physical locations, the detection device can be omnidirectional as it tumbles inside the GI tract. Alternatively, the detection device can be semi-omnidirectional, such as in the case when an anti-tumbling feature, like as a tail or an expanding polymer, is included. This is particularly advantageous when the detection device is passing through larger diameter anatomies, such as the stomach and colon.

The detection device is further configured for efficient management of memory storage and transmission to external devices. Detection of tracer spikes requires far less data storage than other data recordation means, like continuous photography or video. Due to the simplicity of data, its transmission is, likewise, more efficient.

Given these improvements to power management, data capture, data storage, and data transmission, the detection device can be optimized to the smallest possible size. This ensures greater patient compliance associated with device administration. Namely, a smaller device is easier to swallow. Further, by reducing the size of related electrical components, the detection device has optimized capacity for a larger battery, thus increasing overall power capability and extending operational time.

With these improvements in mind, each of the detection device and tracer are described herein, followed by a description of implementation methods for diagnostic screening of the GI tract.

### The Detection Device

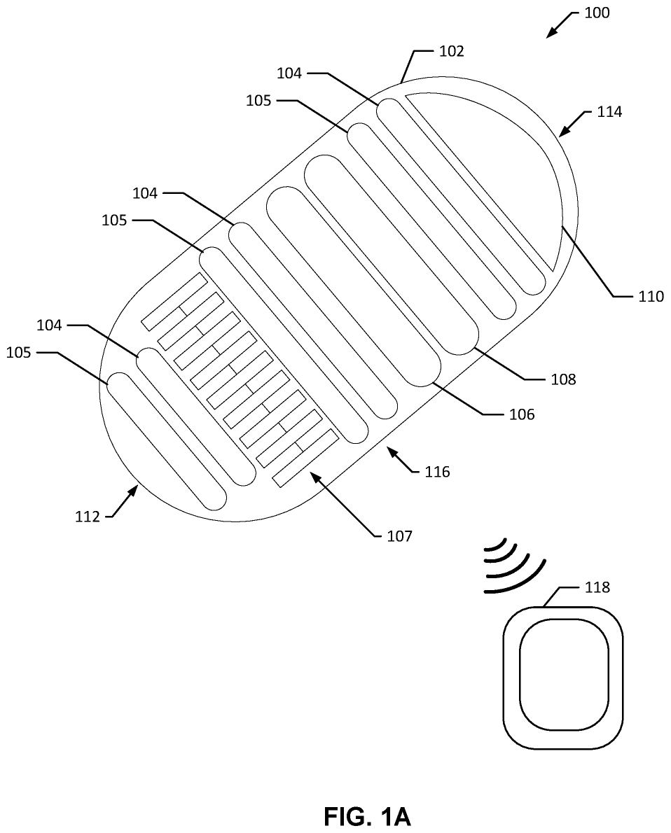

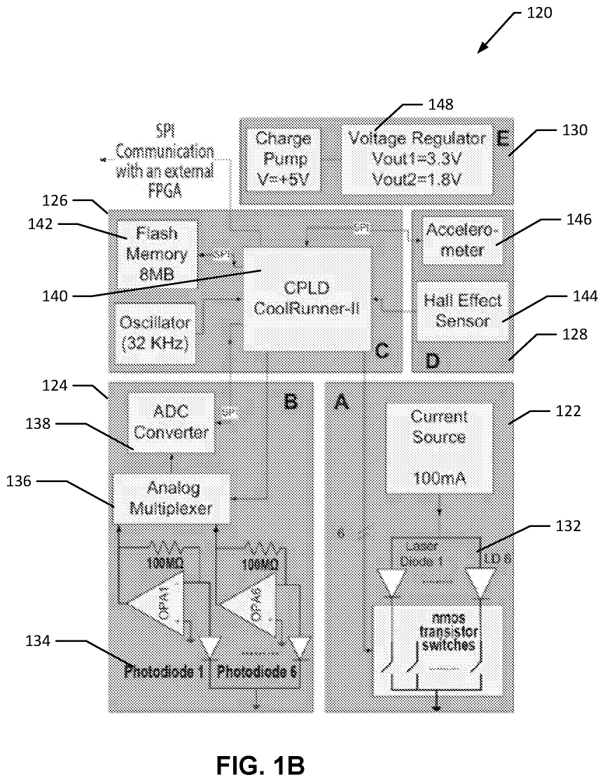

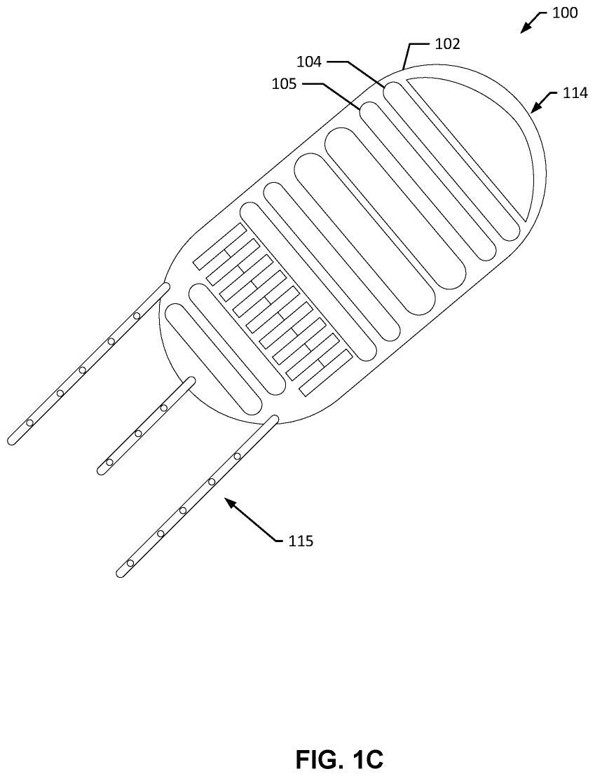

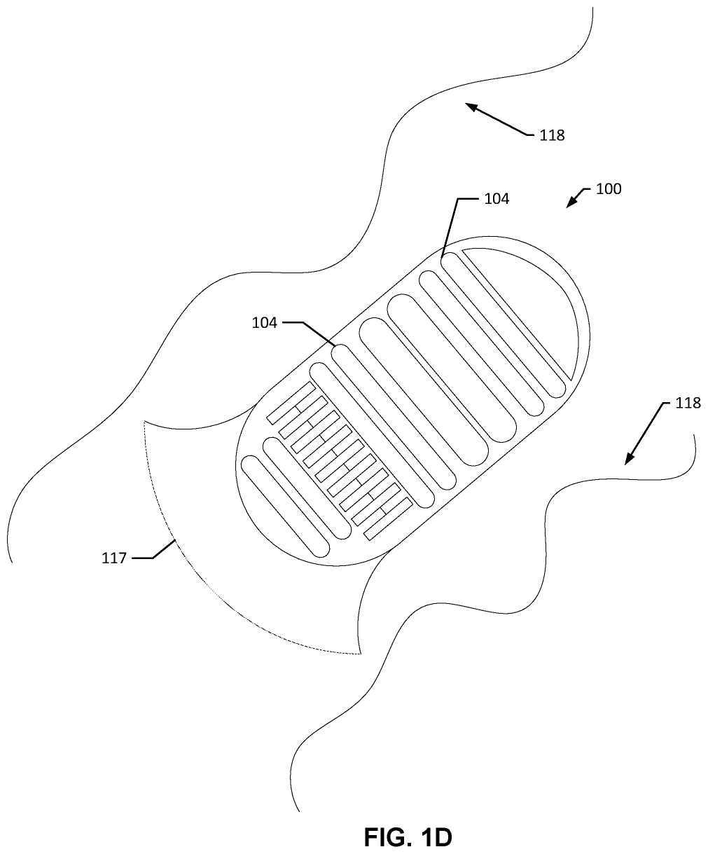

FIG. 1A is an elevation view of a detection device 100, according to an example embodiment of the present disclosure. Detection device 100 includes an enclosed body 102, a plurality of tracer sensors 104, a battery 106, a memory 108, and a transmitter 110. Enclosed body 102 is self-contained, such that the components noted above are hermetically sealed within enclosed body 102.

The enclosed body 102 may be constructed of any suitable plastic material, such as polycarbonate like Makrolon®, polyvinyl chloride (“PVC”), non-DEHP PVC, Krayton polypropylene mixture, or other similar materials. It should be appreciated that enclosed body 102 is constructed of materials that are appropriate for human or large animal ingestion, which will not degrade when passed through a digestive tract. In an embodiment, the enclosed body 102 is transparent or semi-transparent to electromagnetic waves that interact with the tracer, either through fluorescence or through altered reflectivity, which may improve tracer detection capabilities described in greater detail herein. Enclosed body 102 is generally configured for anatomical introduction via swallowing. Thus, enclosed body 102 has a smooth profile and is appropriately dimensioned for a patient's GI tract. For example, enclosed body 102 is approximately 2 to 3 cm long and 1 cm wide. It should be appreciated, however, that smaller and larger enclosed bodies are contemplated.

The plurality of tracer sensors 104 is disposed within enclosed body 102, along with an electronics module 107, that control the tracer stimulation/detection process as well as the storage and data transmission process. Tracer sensors 104 may be disposed at a proximal end 112 of enclosed body 102, a distal end 114 of enclosed body, or along a side portion 116 between proximal end 112 and distal end 114. In the primary embodiment disclosed herein, the plurality of tracer sensors 104 is configured to detect the concentration of fluorescent material, such as a fluorescent tracer, throughout the GI tract. For example, the plurality of tracer sensors 104 includes a plurality of photodiodes along with photonic stimulators 105, such as laser diodes, organic light emitting diodes, or other related illumination means, to illuminate fluorescent material within the GI tract for detection. For example, in an embodiment, the plurality of tracer sensors 104 includes a plurality of laser diodes for illuminating fluorescent material and a plurality of photodiodes for detecting illuminated fluorescent material. This particular embodiment, including both laser diodes and photodiodes, is described in greater detail herein with reference to FIG. 1B.

It should be appreciated, however that other non-fluorescent tracers are contemplated by this disclosure. The alternate methods could include magnetic sensing (detection) and or electromagnetic detection, such as via radiofrequency and microwave detections. The stimulators could be magnetic, for tracer polarization. For radiofrequency detection, stimulation is an electromagnetic (EM) wave and detection is also an EM wave, with properties that are modified by the tracer. Hence, tracer sensors 104 are not, in any way, limited to fluorescence-only detection. For example, tracer sensors 104 may include a radiating antenna and a receiving antenna, to detect permeability of iron oxide particles in a contrast agent including dielectric constant, to detect magnetism in a contrast agent, or any other related means for detecting contrast agents.

Furthermore, it should be appreciated that the plurality of tracer sensors 104 may include additional features or capabilities related to targeted therapy. Namely, in an embodiment, detection device 100 is designed to go beyond screening and diagnosis, and is intended to assist in targeted therapy. Upon detection of high concentrations of the tracer, an electronic process is triggered whereby the detection device 100 uses one of the following modalities: light, hyperthermia, ultrasound, or pH change, to trigger the release of a drug that is loaded within the tracer, at the site of abnormal cells. Thus, in this particular embodiment, detection device 100 may be configured for both identification and treatment purposes.

In an embodiment, FIG. 1A, tracer sensors 104 are omnidirectional, such that the sensors 104 can detect concentration of the tracer, regardless of whether the sensor is facing towards the tracer or facing away from the tracer. Disposing the plurality of tracer sensors 104 in different locations on detection device 100, such as proximal end 112, distal end 114, and side portion 116, or a number of side portions, may improve omnidirectional capabilities.

With the omnidirectional embodiment, because the tracer detection device is searching for significant fluctuations in the tracer concentrations, ideally the detection device 100 should not respond to changes in intensity due to changes in distance from the GI tract walls, in cases where the GI tract is significantly wider that the capsule. To address this undesired effect, in wider GI tract segments, detection device 100 can include a semiconductor-based, miniaturized 3D scanner. Alternatively, optical sensors, interspersed near tracer sensors 104 can approximately estimate the distance between enclosed body 102 and an intestinal wall 118, through measuring the light reflections of diametrically opposite detectors, at a select wavelength, which is not affected by the tracer concentration. By combining that information along with total light reflected, a means for normalizing the background signature of the tracer is established and fluctuations due to high tracer concentrations are identified. Generally, distance detection mechanisms such as those disclosed herein can be used to recalibrate the background intensity levels based on distance from the walls of the adjacent sides.

The battery 106 is likewise disposed within enclosed body 102. Battery 106 is configured to power the plurality of tracer sensors 104. Battery 106 may further power any additional components of detection device 100, such as memory 108 and transmitter 110. In an example embodiment, battery 106 is a silver oxide battery. In an example embodiment, battery 106 is configured to power detection device 100 for the entire duration of a typical GI tract cycle, such as anywhere from 4 to 32 hours.

Memory 108 is likewise disposed within enclosed body 102. Memory 108 is configured to receive measurement values detected by the plurality of tracer sensors 104, such as baseline tracer readings and any tracer spikes, and store these measurement values locally on detection device 100. Memory 108 may be any volatile or non-volatile memory device, such as RANI, ROM, EEPROM, or any other device capable of storing data.

Transmitter 110 is likewise disposed within enclosed body 102. Transmitter 110 is configured to transmit measurement values that are detected by the plurality of tracer sensors 104 (and stored in memory 108) to an external device 118. For example, external device 118 may be a smartwatch, wristband, cellphone, a tablet, a laptop, a medical device, or any other related external device for receiving information from detection device 100. In an example, transmitter 110 sends measurement values to external device 118 via Bluetooth, WiFi, or other related means for wireless communication.

Specifically, information is only transmitted from detection device 100 to external device 118 after detection device 100 exits the body. To identify when detection device 100 has passed through the entire GI tract, and exited the body, detection device 100 may monitor and detect a change in temperature and/or a change in lighting conditions; these changed conditions may indicate that detection device 100 has exited the body, and thus transmission to external device 118 is appropriate. Waiting to transmit data until detection device 100 has fully passed through the entire GI tract and exited the body may advantageously ensure that detection device 100 transmits data to external device 118 only once or only a few times. By reducing the number of data transmissions between detection device 100 and external device 118, detection device 100 can reduce its power consumption (associated with data transmission) and thus reduce its overall size.

Detection device 100, and specifically the plurality of tracer sensors 104, is configured to detect concentration spikes of the tracer. For example, while detection device 100 is travelling across the GI tract, tracer sensors 104 may detect a much higher concentration of the tracer at a given location: this is a tracer spike. The detection device 100 may associate the tracer spike with a discrete location in the GI tract, such as the physical location where the spike was detected and/or a time when the spike was detected tied to typical digestive cycle times. As previously noted, detecting tracer spikes (as opposed to continual photography or videography) may advantageously reduce power requirements of battery 106, may reduce data storage requirements of memory 108, and may reduce transmission requirements of transmitter 110, and thus maximize the battery life. The process of triggering the location estimation process through the detection of the tracer concentration is an additional feature that maximizes battery life, since there is no need to estimate/record locations where tissue is normal.

By providing measurement values, such as spikes, to external device 118, a physician can determine if there is abnormal tissue based off when and where, within the GI tract, the signal was detected. Again, as previously noted, data transmission of tracer signals (as opposed to still images or video) is far more efficient. Efficient data transfer ensures the patient is not required to wear a belt with a data acquisition subsystem for communication with transmitter 110. In certain embodiments, detection device 100 may merely identify surpassing a threshold level of the tracer concentration and subsequently provide the physician with a “endoscopy needed” decision; by comparison, if detection device 100 did not identify surpass a threshold of tracer concentration then the physician would not do any follow-up tests.

In an embodiment, measurement values detected by the plurality of tracer sensors 104 comprise a series of data points including the plurality of signals. For example, the data set is provided by transmitter 110, such that the series of data points can be displayed on external device 118. In this embodiment, a single or several signals can be evaluated by the physician, including both the magnitude of each signal and possibly its discrete location within the GI tract.

In alternate embodiments, detection device 100 further includes at least one camera, configured to temporarily capture pictures or video of the GI tract. In one embodiment, responsive to detecting a tracer spike (via tracer sensors 104), detection device 100 may record visual evidence of the GI tract that can be later associated with the tracer spike. The camera may be a hyperspectral camera, with the capability of capturing images over several different electro-magnetic spectra such as both visible light and infra-red light. Visual evidence, such as photographs and video, may be recorded while illumination device, such as a laser diode, is on. This embodiment is beneficial to provide images of intestinal regions that are otherwise inaccessible to endoscopes, such as the jejunum. In another embodiment, detection device 100 includes at least two cameras for depth estimation and/or 3D image reconstruction. In another embodiment, detection device 100 captures visual evidence with a multi-dot collimator, such as an array of infra-red VCSELs in conjunction with an infra-red camera, for 3D image reconstruction.

In alternate embodiments where detection device 100 includes a camera, such as a hyperspectral camera, detection device 100 may further include a timer, accelerometer, or other related components for determining acceleration and deceleration as detection device 100 passes through the GI tract. Specifically, for example, if detection device is moving faster through particular anatomical structures, such as the small intestine, it would be desirable to capture pictures or video at a higher rate or frequency. Thus, in an example embodiment, detection device 100 calculates its speed or acceleration and adjusts the frequency for capturing photos/video accordingly.

FIG. 1B illustrates a circuit diagram of a detection device, according to an example embodiment of the present disclosure. More particularly, circuit system 120 is an integral part of detection device 100 and is located within enclosed body 102.

Circuit system 120 includes an illumination module 122, a detection module 124, a controller module 126, a sensor module 128, and a power module 130. Illumination module 122 includes a plurality of laser diodes 132, such as six excitation laser diodes biased intermittently in sequence, by a constant current source via six n-MOSFET transistors. Detection module 124 includes a plurality of photodiodes 134, such as six detector photodiodes that include an integrated long-pass filter connected to six operational amplifiers for the amplification of the weak tracer signal. Detection module 124 may further include an analog multiplexer 136 for sequentially selecting and routing each signal to a twelve bit ADC converter 138. In an embodiment, the plurality of laser diodes 132 and the plurality of photodiodes 134 together comprise the plurality of tracer sensors 104.

Controller module 126 includes a CPLD Cool-Runner-II 140 that implements signal-processing algorithms and SPI communication protocols to interface with the peripheral units. Controller module 126 may further include a non-volatile memory chip 142 for storing the digitized tracer data (e.g., memory 108). Sensor module 128 may include a Hall effect sensor 144 and a three-axis accelerometer 146 for sampling rate regulations. Power module 130 includes a voltage regulator 148 communicating with several different voltage supplies for powering the CPLD 140 and the peripheral units (e.g., battery 106).

Generally, because the photocurrent generated from the tracer like fluorescent light is very small, such as pico to nano Amps, the circuit system 120 uses high-gain transimpedance amplifiers (1×10⁸ V/A) with a low input current noise (20 fA/sqrt(Hz)). The digitization is performed via a 12-bit A/D converter, with a reference voltage of 3 V, giving a quantization step of 0.7 mV. Thus, the measurable photocurrent range is 7 pA to 30 nA.

It should be appreciated that circuit system 120 depicted in FIG. 1B above is an exemplary circuit system. In various embodiments, detection device 100 may include circuitry with more, fewer, or different electrical components as generally contemplated by a person having ordinary skill in the art.

Generally, such as in the illustrated embodiment of FIG. 1A, enclosed body 102 is an egg-shaped body. It should be appreciated, however, that alternate geometries for enclosed body 102 are contemplated herein. For example, enclosed body 102 could be cylindrical, spherical, pear-shaped, or any other related geometry. A pear-shaped enclosed body may advantageously include a weight or other feature for self-leveling capabilities as enclosed body 102 passes through the GI tract. Detection device 100 may additionally, or alternatively, include other features for self-leveling capabilities.

For example, FIG. 1C illustrates an elevation view of a tracer detection device with a tumble resisting tail and a semi-omnidirectional detection field. Specifically, detection device 100 may further include a plurality of flexible tails 115. The plurality of flexible tails 115 drag behind enclosed body 102 as enclosed body 102 passes through the GI tract. In this way, the plurality of flexible tails 115 acts as an anti-tumbling feature, thus maintaining the directional orientation of the detection device 100. By maintaining the directional orientation of the detection device 100, the plurality of flexible tails 115 may further ensure that the plurality of tracer sensors 104 is oriented in the proper direction while passing through the GI tract. In an embodiment, the plurality of flexible tails 115 may further electrically detect physical contractions within the GI tract and/or stimulate the GI tract in order to create or inhibit peristalsis. For example, the plurality of flexible tails 115 may include exposed electrode openings, configured to detect or produce different electrical signals along each tail. Each opening may provide for independent recording capability for detection and/or stimulation capability for producing electrical signals. In an embodiment, each electrode opening on each flexible tail 115 communicated with detection device 100 via an individual wire. For example, each flexible tail 115 may include ribbon cable, with openings at different points along tail 115.

Likewise, for example, FIG. 1D illustrates an elevation view of a tracer detection device with a tumble resisting, expandable polymer feature and a semi-omnidirectional detection field. Specifically, detection device 100 may further include an expandable polymer 117. The expandable polymer 117 expands upon entry to a particular anatomical region, such as the large intestine. The expandable polymer 117 is geometrically configured to act as an anti-tumbling feature, thus maintaining the directional orientation of the detection device 100. For example, the expandable polymer 117 ensures that detection device 100 is equidistant from intestinal walls 118. By maintaining the directional orientation of the detection device 100, the expandable polymer 117 may further ensure that the plurality of tracer sensors 104 is oriented in the proper direction while passing through the GI tract.

The Tracer

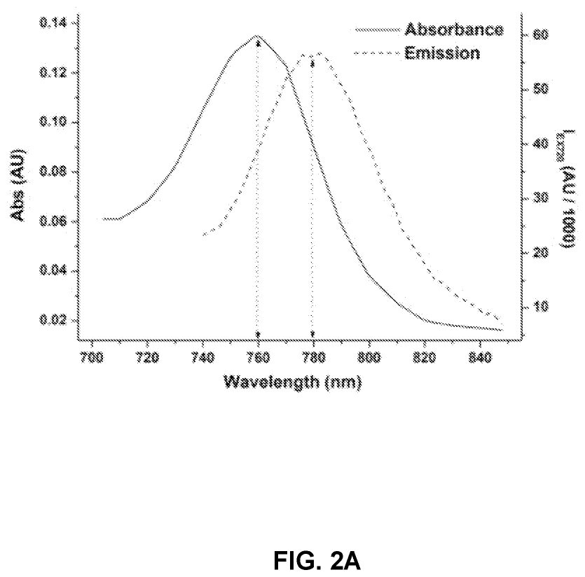

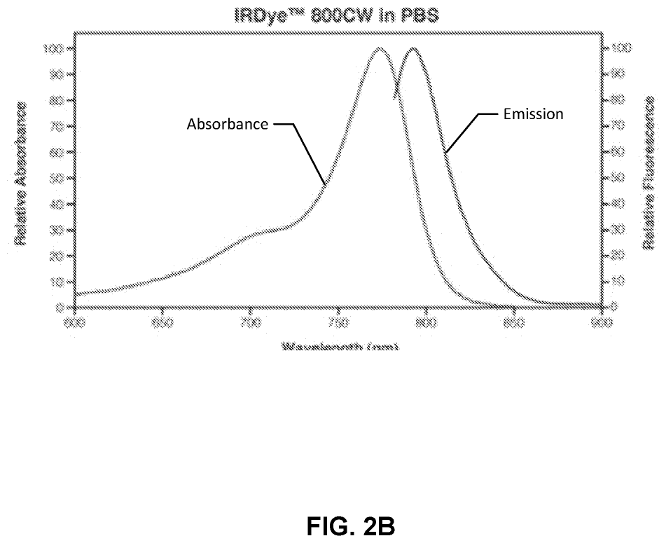

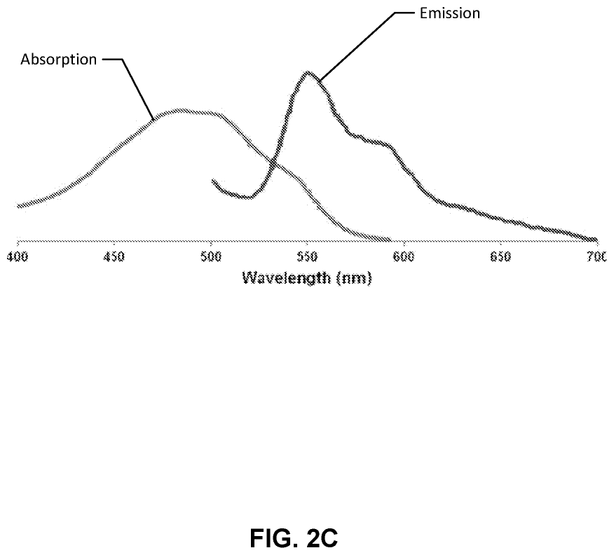

FIG. 2A is a graph of Near-Infra-Red fluorescence absorbance and emission spectra of CF750 at varying wavelengths, according to an example embodiment of the present disclosure. CF750 has a NIRF peak excitation (solid line) and a peak emission (dotted line) at 760 nm and 780 nm, respectively. Likewise, FIG. 2B is a graph of near-infra-red fluorescence absorbance and emission spectra of IRDye® CW800, according to an example embodiment of the present disclosure. IRDye® CW800 has a peak NIRF excitation and a peak NIRF emission at 774 nm and 789 nm, respectively. FIG. 2C is a graph of absorbance and emission spectra of anticancer drug doxorubicin at varying wavelengths, according to an example embodiment of the present disclosure. Doxorubicin has a peak excitation and a peak emission at 470 nm and 550 nm, respectively. As shown in FIGS. 2B and 2C, the drug doxorubicin and the NIRF labelling agent IRDye® CW800 have different absorption and emission maxima characteristics, allowing them to be easily distinguished when combined in a nanoparticle and when spectral analysis is used to select the signal.

In an embodiment, the tracer may be nanoparticles capable of being detected based on NIR, short-wavelength infrared (SWIR)-second near-infrared window, electromagnetic (microwave/radio-frequency (MW/RF)) and magnetic detection that are less affected by the depth of the lesion. The tracer may include NIRF-labeled liposomal nanoparticles, NIRF-labeled polymeric nanoparticles, or iron oxide nanoparticles for MW/RF magnetic sensing. The tracer may carry a drug or API which may be physically entrapped within or covalently conjugated to the nanoparticles. While the embodiments disclosed below primarily refer to NIRF-labeled liposomal nanoparticles, it should be appreciated that other types of tracers are contemplated herein. In a different embodiment, the tracer includes NIRF nanovesicles, such as lipid based quatsomes and other potential tracers based on quatsomes.

In an embodiment of the invention, the tracer is NIRF-labeled liposomal nanoparticles. The NIRF liposomal nanoparticles generally may include a mixture of lipids selected from the group consisting of 1,2-Dipalmitoyl-sn-glycero-3-phosphocholine (DPPC; 16:0 PC), 1,2-Distearoyl-sn-glycero-3-phosphocholine (DSPC 18:0), (w-methoxy-polyethylene glycol 2000)-N-carboxy-1,2-distearoyl-sn-glycero-3-phosphoethanolamine (PEG²⁰⁰⁰-DSPE) and combinations thereof. The NIRF liposome may further include a NIRF labelling agent. The NIRF labelling agent may be a conjugate with a lipid, for example, N,N-Distearylamidomethylamine (DSA), wereby the conjugate has a chemical structure of Formula I; and R is selected from the group consisting of IRDye® 800CW, XenoLightTM CF750, DyLight 680, IRDye® 750, IRDye® 800RS, IRDye® 700DX, ATT0680, ATTO 700, ATTO 725, ATT0740 and DyLight 680 and combinations thereof. The NIRF labelling agent may include IRDye® 800CW-DSA. The NIRF labelling agent may include near-infrared (NIR) nanoparticles.

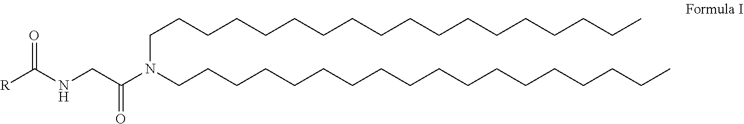

In an embodiment of the invention, the NIRF labeling agent resides in a layer of polymer of the NIRF liposome nanoparticles, whereby the layer of polymer prevents degradative enzymes in the body from cleaving the labeling agent. The NIRF liposome optionally may be loaded with a drug or API. The drug or API may be loaded to the NIRF liposome through different mechanism and may be encapsulated inside the core of the NIRF liposome nanoparticles. For example, the anticancer drug doxorubicin, may be loaded via pH ingredient at a temperature ranging from 37° C. to 42° C. The NIRF liposome particles are stable under normal physiological conditions. The NIRF liposome particles, when loaded with a drug or an API, are stable under normal physiological conditions but may rapid release the encapsulated drug or API when a lipid phase change is triggered or activated by light, hyperthermia, ultrasound, or pH change induced by the detection device 100 or an external source.

In an embodiment, the tracer is a NIRF labeled polymeric nanoparticles derived from hyaluronic acid (HA), with either physically entrapped or covalently conjugated IRDye® CW800. The NIRF polymeric nanoparticles may further include a drug or API for targeted drug delivery to the lesion and treatment of the lesion.

In an embodiment, the tracer is polymer-based nanoparticle formulation using Human Serum Albumin. The Albumin nanoparticles further comprise a NIRF labelling agent. The NIRF labeling agent may be physically entrapped or covalently conjugated IRDye® CW800. The Albumin nanoparticles may optionally comprise a drug or API for targeted therapy of certain diseases.

In an embodiment, the tracer is NIRF-quantum dots (NIRF-QDs).

In an embodiment, the tracer is iron oxide magnetic nanoparticles for electromagnetic such as MW/RF imaging and magnetic sensing. The iron oxide magnetic nanoparticles composition may include (Fe₃O₄-PMA). The iron oxide magnetic nanoparticles formulation may further comprise at least one [(ZnₓFe_{y})Fe₂O₄]-PMA, where the ratio of x to y is from 1:1 to 1:10, preferably 1:1 to 1:6. The iron oxide magnetic nanoparticles formulation may comprise a mixture [(ZnₓFe_{y})Fe₂O_{4]}-PMA at different x/y ratios, such as a mixture of [(Zn_{0.18}Fe_{0.82})Fe₂O₄]-PMA and [(Zn_{0.39}Fe_{0.61})Fe₂O₄]-PMA.

In an embodiment, the tracer is administered to a patient orally or by IV-injection.

In an embodiment, detection device 100 uses two different wavelengths.

### Examples of Preparing the Tracer

A first example includes using NIRF lipid-based nanoparticles as a tracer or a diagnostic chemical entity (DCE). The NIRF liposome nanoparticles are prepared according to the following methods and steps.

Regarding materials and general methods, 1,2-Dipalmitoyl-sn-glycero-3-phosphocholine (DPPC; 16:0 PC) 1,2-Distearoyl-sn-glycero-3-phosphocholine (DSPC 18:0) and (ω-methoxy-polyethylene glycol 2000)-N-carboxy-1,2-distearoyl-sn-glycero-3-phosphoethanolamine (PEG²⁰⁰⁰-DSPE) can be purchased from Avanti Polar Lipids (Alabaster, Ala., USA). CW800-NHS-ester from LI-COR Biotechnology—GmbH (Bad Homburg, Germany). Cell media were from Life Technologies (Carlsbad, CA, U.S.) while other materials were from Sigma-Aldrich (St. Louis, Mo., USA) and are of analytical grade. Other lipids are synthesised as described below. ¹H (400 MHz) NMR spectra can be recorded on a Bruker Advance 400 spectrometer using residual chloroform or dichloromethane as internal standards. Results are reported as chemical shifts in ppm from TMS, with peaks described as s=singlet, br=broad singlet, d=doublet, t=triplet, q=quartet, m=multiplet, and coupling constants J given in hertz (Hz). Mass spectroscopy can be carried out on Thermo LCQ DECA XP or Agilent HP1100 MSD spectrometers depending on availability. Analytical HPLC is carried out using an Agilent 1100 series instrument equipped with a multi-wavelength diode array detector, a 1260 Infinity fluorescence detector, a Polymer Laboratories PL-ELS-2100 evaporative light scattering detector, and a 5 cm Hypersil C18 5μm reverse-phase column. Synthesised lipids are analysed using gradient: 0 min, 100% water, 2.5 mL/min; 1 min, 100% water; 11 min, 100% ACN; 11 min, 100% ACN; 23 min, 100% methanol; 25 min, 100% methanol; 27 min, 100% water, 1.8 mL/min; 30 min, 100% water, 2.5 mL/min and showed purity at least 95%. Thin Layer Chromatography (TLC) was carried out on F254 silica gel 60 plates, with spots visualised by UV illumination or vanillin/ninhydrin staining and developed with a heat gun. Flash column chromatography was performed on 40-63 μm silica gel.

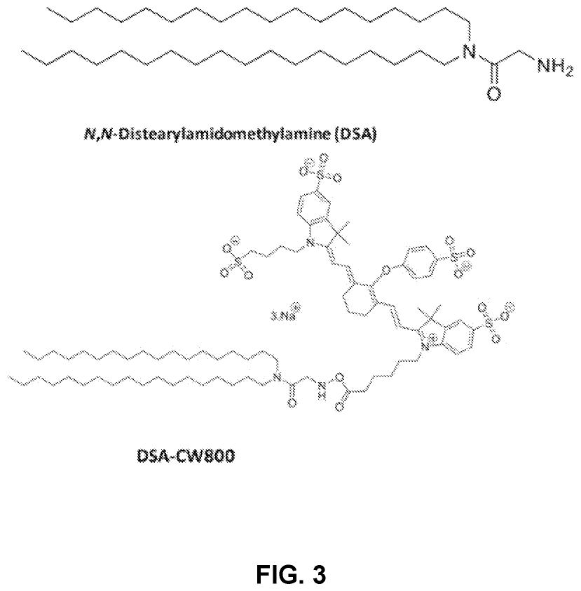

FIG. 3 is an illustration of exemplary chemical structures of lipid N,N-Distearylamidomethylamine (DSA) and its conjugate with IRDye® 800CW (or CW800), according to an example embodiment of the present disclosure.

Regarding synthesis of lipids, N,N-Distearylamidomethylamine (DSA) is synthesised according to previous methods (Bioconjugate Chem. 2008, 19, 1, 118-129). H (400 MHz; CD₂Cl₂; 296 K) δ 3.84 (s, 2H, OCCH₂NH₂), 3.29 (t, J=8.0 Hz, 2H, OCNCH₂), 3.11 (t, J=7.8 Hz, 2H, OCNCH₂), 1.50 (m, 4H, OCNCH₂CH₂), 1.25 (s, 60H, alky chain CH2), 0.88 (t, J=6.3 Hz, 6H, CH₃). ¹³C (100 MHz; CD₂Cl₂; 296 K) δ 166.6 (OCN) 48.8 & 48.1 (OCNCH₂), 41.6 (OCCH₂NH₂), 31.1 (CH₃CH₂CH₂), 30.9-30.8 (alkyl chain CH2), 29.9 (OCNCH₂CH₂CH₂CH₂), 28.8-28.3 (OCNCH₂CH₂CH₂), 24.2 (CH₃CH₂), 15.4 (CH₃). TLC (15% MeOH in CH₂Cl₂ with 0.5% NH₃) gave R_{f}0.55 with the DSA spot showing red after sequential vanillin and ninhydrin stains. HPLC t_{R}=13.3 min; ESI-MS [M+H]⁺579.7 m/z (expect 578.6 m/z for C₃₈H₇₈N₂O).

DSA (3 mg) is mixed with 800 μL of 1:1 dichloromethane and methanol in a glass vial. IRDye® 800CW NHS ester (0.5 mg) was dissolved in methanol (200 μL) then immediately combined with the lipid solution. The mixture is covered to protect it from light and left on a vortex mixer for 2.5 h. The solvent is then reduced in vacuum, dissolved in minimal amount of chloroform and applied to a silica micro-column (˜3 mL) also in chloroform. The unbound fraction is reapplied several times, before elution and washing with the same solvent. Excess DSA and unreacted dye material is retained on the column and the latter is estimated at <10% of the coloured fraction. The unbound product is dried to a film and stored as a solution in 1 mL chloroform. This blue/green solution shows a strong fluorescence emission with Exₘₐₓ 785 nm and Emₘₐₓ 808 nm (reported values of 774 nm and 789 nm for the free dye).

Regarding preparation of NIRF liposome nanoparticles, a mixture (20 mg total lipid) of DPPC (85.3 mol %), DSPC (9.7 mol %), and DSPE-PEG²⁰⁰⁰-MeO (5 mol %) is combined from chloroform/methanol stock solutions. IRDye® 800CW-DSA (˜60 μg) is added and the mixture dried to a film in vacuo using a small, round-bottomed flask. This is hydrated with 20 mM HEPES buffer and treated 5 times by freeze/thaw in liquid nitrogen and hot water to fragment the film. The resulting suspension is then sonicated at 60° C. for 10 min forming a milky colored (blue/green) suspension, before extrusion at least 3 times through a 100 nm membrane using a Northern Lipids (Burnaby, Canada) LIPEX extruder heated to 55° C. and pressurized to about 10-20 bar. The external buffer is then exchanged to sterile 20 mM HEPES pH 7.4 with 5% glucose (w/v) using a PD10 size exclusion column (Amersham, Buckinghamshire, UK). The resulting clear preparation is applied to a PD-10 column, washed and eluted with the same buffer. The single colored fraction eluted within 3-4 mL and is shown to have a Zavg size of 97 nm and PDI of 0.21 using a Malvern Nanosizer.

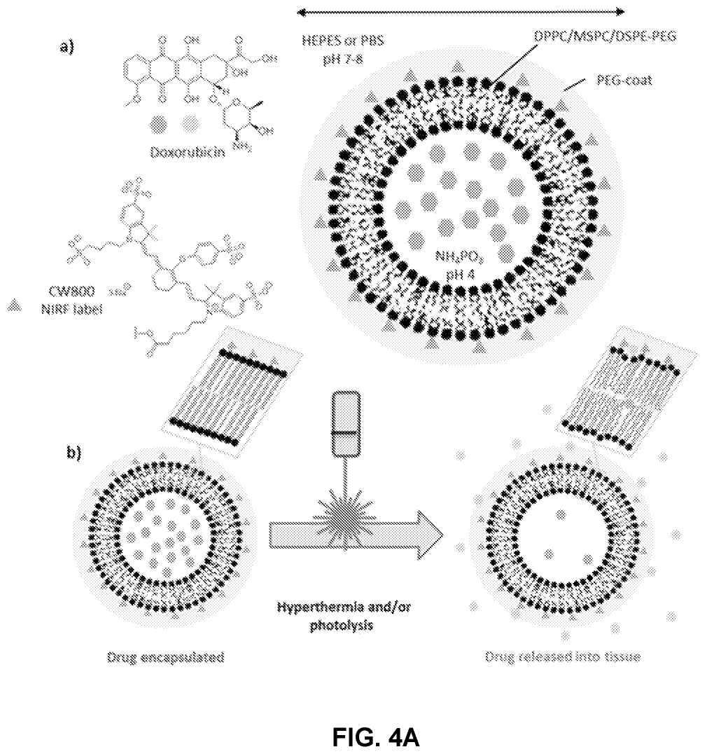

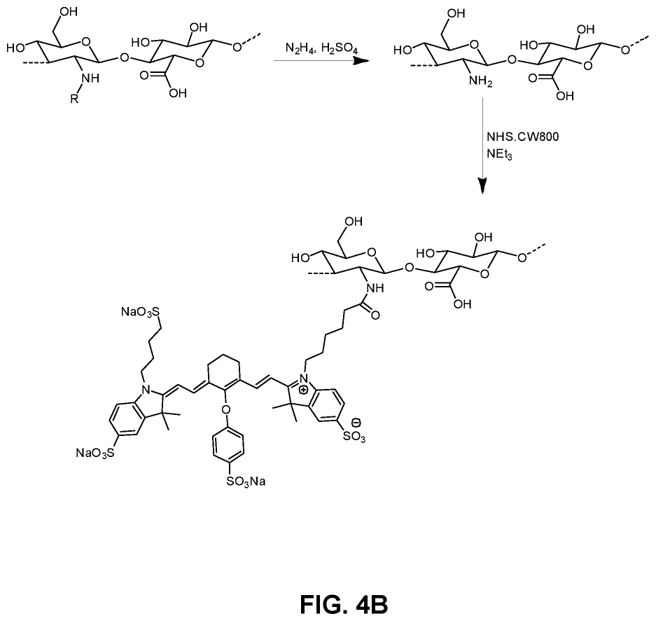

FIG. 4A illustrates a typical scheme of liposome structure with NIRF labeling agent IRDye® CW800, according to an example embodiment of the present disclosure. For example, the liposome envelope is formed of neutral phospholipids, covered in a PEG stabilizing coat and decorated with a NIRF label of DSA-CW800; a) a drug such as doxorubicin can be loaded via pH gradient at 38° C.; and b) the resulting nanoparticle is “stable” under normal physiological conditions but a lipid phase change rapidly releases the encapsulated drug when surrounding tissue is radiated by light. Nanoparticles trapped by intestinal lesions can have their drug release activated by light emitted by the detection device 100.

A second example includes using NIRF polymeric nanoparticles as a tracer. The NIRF polymeric nanoparticles are NIRF labeled hyaluronic acid and nanoparticles, which is prepared by a process illustrated in FIG. 4B. In the second example, the NIRF includes hyaluronic acid and nanoparticles. In a third example, the NIRF includes Albumin nanoparticles with CW800. In a fourth example, the NIRF includes Quantum Dots emitting at the NIRF.

In a fifth example magnetic iron oxide nanoparticles for MW-RF imaging and magnetic sensing is implemented. Namely, this example includes the synthesis of ferrites by thermal decomposition. 0.706 g of tris(acetylacetonato) iron (III) (Fe(acac)3) and 2.58g of hexadecandiol were added to a 50 mL three neck round bottom flask along with (2.11 mL) oleic acid, (2.82 mL) oleylamine and (20 mL) benzyl ether. The mixture is then stirred and heated to 200° C. for one hour and 300 ° C. for two hours under nitrogen. After the reaction is cooled to room temperature, the mixture is added to two 50 mL falcon tubes with 40 mL of ethanol. Falcon tubes are centrifuged for 30 minutes at 4000 RPM. Ethanol, i.e. the supernatant was discarded. The pellet left in the falcon tube is resuspended in 20 mL of hexane, and 50 μL of oleic acid and oleylamine are added. The solutions are combined into only one falcon tube. The tube is centrifuged for 10 minutes at 4000 RPM. The supernatant is kept, and the pellet is discarded. Then 20 ml of ethanol is added to the falcon tube and is centrifuged for 30 minutes at 4000 RPM. The supernatant is discarded, and the pellet is left to dry under vacuum overnight. Fe₃O₄ is synthesized by the steps above. The (ZnₓFe_{y}) Fe₂O₄ is synthesized following the same steps as shown above with the first step modified for each composition. The feeding material of the first step is modified to be a combination of FeCl₂, Fe(acac)₃ and ZnCl₂ at different ratios.

PMAO (Polymaleic anhydride-alt-1-octadecene) is dissolved in chloroform in a round bottom flask and left to stir vigorously for 1 minute. After a clear solution is obtained, 2 mg of SPIONs are added to the flask. The solution is then left to stir with a magnetic stirrer for 1 hour at room temperature in the sealed flask, until a translucent solution with a red-brown tint is produced. The flask is then placed in a rotary evaporator for 20 minutes (set to room temperature) to separate the chloroform. Chloroform (1 mL) is then added to the flask to re-dissolve the SPIONs followed by of NaOH in water. The flask is continually agitated whilst left on a hot plate set to 60° C. After ten minutes, a further 10 mL of NaOH is added whilst still heating and agitating the flask. This is done until a clear black solution showing no evidence of a biphasic system is produced. The solution is poured into two centrifugal filter units and centrifuged for 15 minutes. The nanoparticles are removed from the filter with distilled water and pipetted in new glass vials.

### Examples of In Vivo Drug Delivery Kinetics and Tumor Growth Suppression

Lipid-based nanoparticles have been proven to be versatile systems for drug delivery. However, suboptimal drug delivery is the main cause of serious side effects or failure of multiple cancer therapies. Drug such as doxorubicin release from the liposomal nanoparticles during their circulation in blood could lead to unwanted toxicity. Inventors of the present claimed invention have identified the need for further development of optically labeled liposomes for image guidance targeted drug delivery and triggered drug release for treatment of certain diseases, such as cancers and tumors.

Inventors have developed NIRF liposomal nanoparticles labeled with IRDye® CW800 and loaded with a drug doxorubicin as illustrated in FIG. 4A. These labeled liposomal nanoparticles allow simultaneous, real-time diagnostic imaging of nanoparticle bio-distribution using MR fluorophore (IRDye® CW800) coupled to a lipid component (DSA) of the lipid bilayer. When combined with multispectral fluorescence analysis, this also allows specific and high sensitivity tracking of nanoparticles in vivo.

The selection of drug and the NIRF label IRDye® CW800 has been based on their different emission spectra characteristics and strong pH dependent variations in doxorubicin absorption and emission spectra characteristics. FIGS. 2B and 2C show the spectra characteristics of the drug doxorubicin and CW800 respectively, which demonstrate that CW800 excitation and emission maxima are different from those of doxorubicin, allowing them to be easily distinguished using multispectral analysis. The liposome formulation includes liposome envelope (liposome bilayers) formed of a mixture of neutral phospholipids of DPPC:DSPC:DSPE-PEG²⁰⁰⁰-MeO at 85.3:9.7:5 (mol/mol). The liposome envelope is covered in a PEG stabilizing coat and decorated with a NIRF label. The NIRF label is IRDye 800CW-DSA in an amount of 0.01% by weight of the total phospholipids. The drug is loaded to the liposome via pH gradient.

As shown in FIG. 4A, the protocol used for doxorubicin loading takes advantages of the effects of different pH buffers inside and outside the liposomes. By forming a PEGylated liposome with an internal aqueous core at about pH 4.0 but using a HEPES or PBS external buffer at about pH 7-8, the resulting pH gradient promotes doxorubicin entrapment within the liposome cavities on incubation at the temperature of about 38° C. The resulting nanoparticle is stable under normal physiological conditions but a lipid phase change rapidly releases the encapsulated drug when surrounding tissue is radiated by light.

Owing to the enhanced permeability retention (EPR) effect, the drug-loaded liposomal nanoparticles accumulate into well vascularized tumors, resulting in an enhanced local concentration. The liposomal nanoparticles trapped by intestinal lesions can have their drug release activated by light emitted by the capsule, thus achieve effective image guidance targeted drug delivery and triggered drug release for treatment of intestinal tumors.

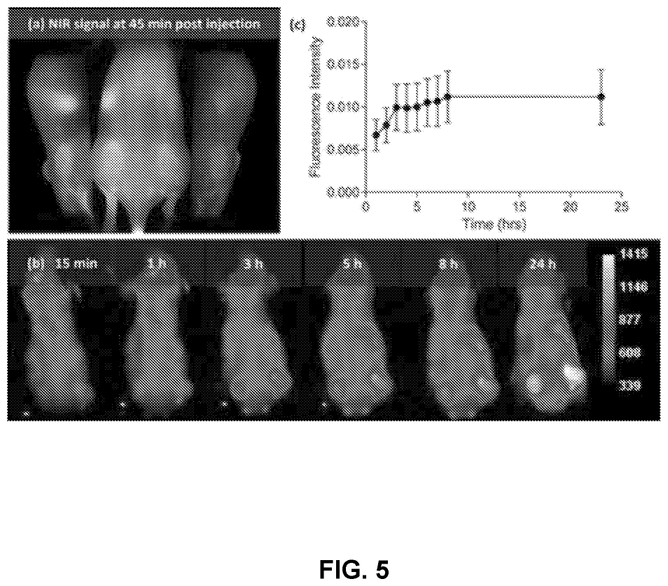

The NIRF labeled liposomal nanoparticles are administered by iv injection and the nanoparticles kinetics were then analyzed as a function of time. It is surprisingly and unexpectedly found that the NIRF labeled liposomal nanoparticles can be imaged clearly in circulation from about 5 min post-injection as the nanoparticles reached and collected within the two flank tumor (FIG. 5A). This accumulation of nanoparticles is monitored over time and shown to promote a distinct increase in the CW800-DSA fluorescence signal (see FIG. 5B), using the following instrument settings: excitation band pass 704 nm (684 to 729 nm effectively) and emission long pass 745 nm, and images acquired over 740 to 950 nm in 10 nm steps. The signal apparent in the neck/head of the animal is considered to be due to the presence of the nanoparticles in the vasculature, in particular the subcutaneous vessels, and not due to uptake to the brain. The maximum signal occurs 15-30 min post-injection and decays rapidly over the following hours, which supports this hypothesis. Conversely, the NIRF signal in the liver and spleen increases over time and has been confirmed by ventral imaging. Semi-quantification of images based on the fluorescence intensities of the tumor as a function of time provides the illustrated nanoparticle kinetic profile (FIG. 5C). The pharmacokinetics are based on the intensity of the NIRF signal coming from the liposomes. The data indicate that signal is gradually increasing during the first 5 h while minimum changes at the fluorescent signal appear from 5 to 24 h. These results were obtained from real-time imaging of nanoparticle tumor accumulation. Detecting the level on nanoparticles in tumors in real time is an important factor for the success of image guided triggered drug delivery

It is surprisingly and unexpectedly observed that in situ drug release from tumor-localized nanoparticles is triggered upon activation by light emitted by the capsule. An apparent increase in drug fluorescence signal is detected within 5 min from the beginning of light emission from the capsule 100. This is considered to correspond with heat-triggered drug release into tumor environments in accordance with the biophysical drug release of the NIRF labeled liposomal nanoparticles of the presently claimed invention.

The observation is also found to correlate agreeably with enhanced reductions in the growth of tumors when treated with the NIRF labeled liposomal nanoparticles loaded with the drug doxorubicin which is specifically delivered to the targeted tumors and released locally by light activation through the capsule 100, in comparison to the control situation where light activation treatment is withheld.

FIGS. 5A, 5B and 5C illustrate examples of in vivo nanoparticles kinetics in tumors generated with IGROV-1 cells subcutaneously implanted to the flanks of SHO mice. FIG. 5A is an illustration of the highlighted vasculature due to nanoparticles presence 45 min after injection. FIG. 5B is an illustration of the accumulation of nanoparticles in tumors over time, in one representative animal. The images are acquired using the same MR acquisition setting at each time point; images are unmixed using the Maestro software, quantified using pixel intensity, and false colored using ImageJ. FIG. 5C shows average signal intensity corresponding to the particle kinetics over time using the quantified images of each acquisition time point. The measurements are an average of 8 tumors with the error bars ±SD.

It is surprisingly and unexpectedly found the NIRF labeled liposomal nanoparticles prepared in this embodiment enabled specific and high sensitivity imaging and tracking of the bio-distribution of the nanoparticles in vivo after injection, had sufficient levels of stability in biological fluids under physiological conditions from the point of administration to the target tissue (namely tumors in this experiment), are effective at delivering the drug to the target tumors area, and effectively release the drug locally upon activation of the light emitted by the capsule 100, when combined with multispectral analysis, as demonstrated in the experimental data in FIGS. 5A to 5C.

### Method of Implementation and Detection

A typical method of implementation and detection will involve both the detection device 100 and the tracer, each of which have been previously described in greater detail above. The method may begin by injecting the tracer into a circulatory system of the patient, such as via intravenous injection, via infusion pump, or by other related means. Alternatively, the tracer may be introduced via digestion.

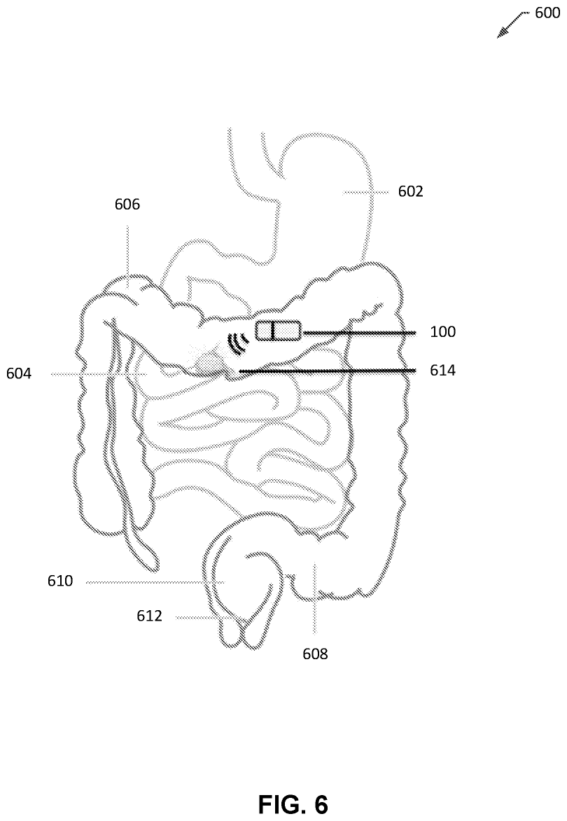

Once the tracer is properly absorbed into the patient's body, detection device 100 is introduced into the GI tract of the patient. Typically, introduction involves the patient swallowing the detection device 100. FIG. 6 illustrates an anatomical diagram of detection device 100 passing through a GI tract 600, after it has been swallowed. GI tract 600 includes a stomach 602, a small intestine 604, a large intestine 606 (including a colon), a sigmoid colon 608, a rectum 610, and an anus 612. Detection device 100 passes along GI tract 600 in the expected digestive direction: from stomach 602 to anus 612.

As illustrated, while proceeding along GI tract 600, detection device 100 detects measurement values, including baseline tracer measurements. In the illustrated embodiment, the tracer is a fluorescent material. For example, the plurality of laser diodes 132 temporarily illuminate the GI tract 600, which triggers any fluorescent material to glow; the plurality of photodiodes 134 detect the fluorescent material as it glows. Detection device 100 passes a GI abnormality 614. For example, the GI abnormality 614 may be a cancerous or pre-cancerous tumor. Detection device 100 detects additional fluorescence measurement values, including a higher concentration of fluorescence, such as near-infra-red fluorescence liposomes. In practice, the plurality of photodiodes 134 detect a higher concentration of fluorescent material through a more intense or brighter glow. In this way, detection device 100 effectively identifies a tracer spike of the tracer, which is associated with the GI abnormality 614.

Detection device 100 then transmits measurement values, including any baseline fluorescence measurements and any measured tracer spikes, to an external device (such as external device 118). In a specific embodiment, device 100 transmits measurement values after exiting through the end of GI tract 600.

The plurality of tracer spikes may be associated with a plurality of discrete locations in GI tract 600, to identify specifically where a GI abnormality 614 exists. Once the discrete physical location of a GI abnormality 614 is known, the abnormality can be monitored or evaluated through further means, such as directed endoscopy.

As used in this specification, including the claims, the term “and/or” is a conjunction that is either inclusive or exclusive. Accordingly, the term “and/or” either signifies the presence of two or more things in a group or signifies that one selection may be made from a group of alternatives.

Without further elaboration, it is believed that one skilled in the art can use the preceding description to utilize the claimed inventions to their fullest extent. The examples and embodiments disclosed herein are to be construed as merely illustrative and not a limitation of the scope of the present disclosure in any way. It will be apparent to those having skill in the art that changes may be made to the details of the above-described embodiments without departing from the underlying principles discussed. In other words, various modifications and improvements of the embodiments specifically disclosed in the description above are within the scope of the appended claims. For example, any suitable combination of features of the various embodiments described is contemplated. Note that elements recited in means-plus-function format are intended to be construed in accordance with 35 U.S.C. § 112 ¶ 6. The scope of the invention is therefore defined by the following claims.

1-31. (canceled)

32. A tracer detection device, comprising:
an enclosed body;
a plurality of tracer sensors, disposed within the enclosed body and configured to detect measurement values both at a surface and underneath the surface of a gastrointestinal tract;
a battery, disposed within the enclosed body and configured to power the plurality of tracer sensors;
a memory, disposed within the enclosed body and configured to receive measurement values detected by the plurality of tracer sensors; and
a transmitter, disposed within the enclosed body and configured to transmit measurement values detected by the plurality of tracer sensors to an external device after the enclosed body has passed entirely through the gastrointestinal tract,
wherein the enclosed body further includes a steering feature that ensures the enclosed body is oriented in an intended direction while passing through the gastrointestinal tract,
wherein the plurality of tracer sensors is further configured to trigger release of a drug within the gastrointestinal tract, and
wherein the plurality of tracer sensors further estimate distances between the enclosed body and walls of the gastrointestinal tract for normalizing signals.

33. The tracer detection device of claim 32, wherein the plurality of tracer sensors are configured to detect a plurality of tracer spikes of a tracer.

34. The tracer detection device of claim 33, wherein the plurality of tracer spikes are associated with a plurality of discrete locations in the gastrointestinal tract.

35. The tracer detection device of claim 33, wherein the measurement values detected by the plurality of tracer sensors comprise a series of data points including the plurality of spikes.

36. The tracer detection device of claim 32, wherein the steering feature is at least one of a long flexible tail and an expandable polymer.

37. The tracer detection device of claim 32, further comprising a hyperspectral camera, configured to selectively capture pictures or video of the gastrointestinal tract when triggered by a tracer signal.

38. The tracer detection device of claim 32, wherein the plurality of tracer sensors trigger release of the drug within the gastrointestinal tract via at least one of light, hyperthermia, ultrasound, or pH change.

39. A tracer lesion detection system, comprising:
a tracer; and
a detection device, including:
a plurality of tracer sensors, configured to detect measurement values both at a surface and underneath the surface of a gastrointestinal tract;
a battery, configured to power the plurality of tracer sensors;
a memory, configured to receive measurement values detected by the plurality of tracer sensors; and
a transmitter, configured to transmit measurement values detected by the plurality of tracer sensors to an external device after the enclosed body has passed entirely through the gastrointestinal tract,
wherein the detection device further includes a steering feature that ensures the detection is oriented in an intended direction while passing through the gastrointestinal tract,
wherein the plurality of tracer sensors is further configured to trigger release of a drug within the gastrointestinal tract, and
wherein the plurality of tracer sensors further estimate distances between the enclosed body and walls of the gastrointestinal tract for normalizing signals.

40. The tracer lesion detection system of claim 39, wherein the plurality of tracer sensors is configured to detect a plurality of tracer spikes of the tracer.

41. The tracer lesion detection system of claim 40, wherein the measurement values detected by the plurality of tracer sensors comprise a series of data points including the plurality of spikes.

42. The tracer lesion detection system of claim 39, wherein the steering feature is at least one of a long flexible tail and an expandable polymer.

43. The tracer lesion detection system of claim 39, further comprising a hyperspectral camera, configured to selectively capture images or video of the gastrointestinal tract when triggered by a tracer signal.

44. The tracer lesion detection system of claim 39, wherein the plurality of tracer sensors trigger release of the drug within the gastrointestinal tract via at least one of light, hyperthermia, ultrasound, or pH change.

45. The tracer lesion detection system of claim 39, wherein the tracer includes at least one of near-infrared fluorescence (NIRF) labeled liposomal nanoparticles or NIRF nanovesicles.

46. The tracer lesion detection system of claim 45, wherein the NIRF-labeled liposomal nanoparticles comprises a mixture of lipids selected from the group consisting of 1,2-Dipalmitoyl-sn-glycero-3-phosphocholine (DPPC; 16:0 PC), 1,2-Distearoyl-sn-glycero-3-phosphocholine (DSPC 18:0), (w-methoxy-polyethylene glycol 2000)-N-carboxy-1,2-distearoyl-sn-glycero-3-phosphoethanolamine (PEG²⁰⁰⁰-DSPE) and combinations thereof, and a NIRF labelling agent selected from the group consisting of IRDye® 800CW-DSA, near-infrared (NIR) nanoparticles, and combinations thereof, wherein the IRDye® 800CW-DSA is a conjugate of IRDye® 800CW with a lipid of N,N-Distearylamidomethylamine (DSA).

47. The tracer lesion detection system of claim 46, wherein the NIRF-labeled liposomal nanoparticles further comprises a drug and/or an active pharmaceutical ingredient (API).

48. The tracer lesion detection system of claim 47, wherein the drug or active pharmaceutical ingredient (API) is chemotherapeutic such as doxorubicin.

49. The tracer lesion detection system of claim 39, wherein the tracer is NIRF labeled polymeric nanoparticles, wherein the NIRF labeled polymeric nanoparticles are selected from: IRDye® 800CW labeled hyaluronic acid nanoparticles, and IRDye® 800CW labeled Albumin nanoparticles.

50. The tracer lesion detection system of claim 39, wherein the tracer is administered to a patient orally or by intravenous (IV) injection.

51. A tracer detection method, comprising:
injecting a tracer into a circulatory system of a patient;
introducing a detection device into a gastrointestinal tract of the patient, wherein introduction is performed by the patient swallowing the detection device;
detecting, via the detection device, measurement values including a plurality of tracer spikes of the tracer both at a surface and underneath the surface of the gastrointestinal tract;
transmitting, via the detection device, the measurement values including the plurality of tracer spikes to an external device after the enclosed body has passed entirely through the gastrointestinal tract, such that the plurality of tracer spikes may be associated with a plurality of discrete locations in the gastrointestinal tract,
wherein the detection device further includes a steering feature that ensures the detection is oriented in an intended direction while passing through the gastrointestinal tract,
wherein the plurality of tracer sensors is further configured to trigger release of a drug within the gastrointestinal tract, and
wherein the plurality of tracer sensors further estimate distances between the enclosed body and walls of the gastrointestinal tract for normalizing signals.
